SEQUENCE LISTING (26 sequences)

SEQ ID NO: 1 - protein, 4 aa, Homo sapiens
  MISC_FEATURE (4)..(4): Amidated Phenylalanine
YPWF

SEQ ID NO: 2 - protein, 4 aa, Homo sapiens
  MISC_FEATURE (4)..(4): Amidated Phenylalanine
YPFF

SEQ ID NO: 3 - protein, 4 aa, Artificial Sequence
  Synthetic mu-opiate receptor peptides
  MISC_FEATURE (4)..(4): Hydroxylated Phenylalanine
YPWF

SEQ ID NO: 4 - protein, 4 aa, Artificial Sequence
  Synthetic mu-opiate receptor peptides
  MISC_FEATURE (4)..(4): Hydroxylated Phenylalanine
YPFF

SEQ ID NO: 5 - protein, 4 aa, Artificial Sequence
  Synthetic mu-opiate receptor peptides
  MISC_FEATURE (4)..(4): Amidated Phenylalanine
YPWF

SEQ ID NO: 6 - protein, 4 aa, Artificial Sequence
  Synthetic mu-opiate receptor peptides
YPFF

SEQ ID NO: 7 - protein, 4 aa, Artificial Sequence
  Synthetic mu-opiate receptor peptides
  MISC_FEATURE (3)..(3): Nitrosylated Tryptophan
  MISC_FEATURE (4)..(4): Amidated Phenylalanine
YPWF

SEQ ID NO: 8 - protein, 4 aa, Artificial Sequence
  Synthetic mu-opiate receptor peptides
  MISC_FEATURE (3)..(3): Nitrosylated Phenylalanine
  MISC_FEATURE (4)..(4): Amidated Phenylalanine
YPFF

SEQ ID NO: 9 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 10 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 11 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 12 - protein, 4 aa, Artificial Sequence
  Synthetic mu-opiate receptor peptides
  MISC_FEATURE (3)..(3): Alkylated Phenylalanine
  MISC_FEATURE (4)..(4): Amidated Phenylalanine
YPFF

SEQ ID NO: 13 - protein, 4 aa, Artificial Sequence
  Synthetic mu-opiate receptor peptides
  MISC_FEATURE (2)..(4): Cyclic Peptide
YKWF

SEQ ID NO: 14 - protein, 4 aa, Artificial Sequence
  Synthetic mu-opiate receptor peptides
  MISC_FEATURE (2)..(4): Cyclic Peptide
YKFF

SEQ ID NO: 15 - protein, 4 aa, Artificial Sequence
  Synthetic mu-opiate receptor peptides
  MISC_FEATURE (2)..(4): Cyclic Peptide
  MISC_FEATURE (2)..(2): Ornathine
YXWF

SEQ ID NO: 16 - protein, 4 aa, Artificial Sequence
  Synthetic mu-opiate receptor peptides
  MISC_FEATURE (2)..(4): Cyclic Peptide
  MISC_FEATURE (2)..(2): Ornathine
YXFF

SEQ ID NO: 17 - protein, 4 aa, Artificial Sequence
  Synthetic mu-opiate receptor peptides
  MISC_FEATURE (2)..(4): Cyclic Peptide
  MISC_FEATURE (3)..(3): Nitrosylated Tryptophan
YKWF

SEQ ID NO: 18 - protein, 4 aa, Artificial Sequence
  Synthetic mu-opiate receptor peptides
  MISC_FEATURE (2)..(4): Cyclic Peptide
  MISC_FEATURE (3)..(3): Nitrosylated Phenylalanine
YKFF

SEQ ID NO: 19 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Nitrosylated Tryptophan
YXWF

SEQ ID NO: 20 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Nitrosylated Phenylalanine
YXFF

SEQ ID NO: 21 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 22 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Alkylated Phenylalanine
YXFF

SEQ ID NO: 23 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 24 - protein, 4 aa, Artificial Sequence
  Synthetic mu-opiate receptor peptides
  MISC_FEATURE (2)..(4): Cyclic Peptide
  MISC_FEATURE (2)..(2): Ornathine
  MISC_FEATURE (3)..(3): Alkylated Phenylalanine
YXFF

SEQ ID NO: 25 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

SEQ ID NO: 26 - protein, 4 aa, Artificial Sequence
  Synthetic mu-opiate receptor peptides
  MISC_FEATURE (2)..(4): Cyclic Peptide
  MISC_FEATURE (3)..(3): Alkylated Phenylalanine
YKFF

(19) United States
(12) Patent Application Publication
(10) Pub. No.: US 2018/0251490 A1
(43) Pub. Date: Sep. 6, 2018

(54) ADVANTAGEOUS MU-OPIATE RECEPTOR PEPTIDE COMPOUNDS

(71) Applicant: Cytogel Pharma, LLC, Darien, CT (US)

(72) Inventor: Theodore E. Maione, Green Island, NY (US)

(21) Appl. No.: 15/981,503

(22) Filed: May 16, 2018

Related U.S. Application Data
(63) Continuation of application No. 15/168,158, filed on May 30, 2016, now abandoned.
Application No. 15/168,158 is a continuation of application No. 12/559,994, filed on Sep. 15, 2009.

(51) Int. Cl. C07K 5/12; C07K 5/107; A61K 9/08; A61K 38/00

(52) U.S. Cl. CPC C07K 5/126; A61K 38/00; A61K 9/08; C07K 5/1016

(57) ABSTRACT

The subject invention provides advantageous new salts of mu-opiate receptor peptides. These salts have been found to have excellent properties in terms of their activity.

### CROSS-REFERENCE TO A RELATED APPLICATION

The subject application is a continuation of co-pending application Ser. No. 15/168,158, filed May 30, 2016; which is a continuation of co-pending application Ser. No. 12/559,994, filed on Sep. 15, 2009, which is incorporated herein by reference its entirety.

The Sequence Listing for this application is labeled “Asfiled_ST25.txt”, which was created on Sep. 15, 2009, and is 8 KB. The entire content is incorporated herein by reference in its entirety.

### BACKGROUND OF INVENTION

### Field of the Invention

This invention relates to salts, and the free base, of peptides that bind with high affinity and selectivity to the mu (morphine) opiate receptor; pharmaceutical preparations containing an effective amount of the compounds; and methods for providing analgesia, relief from gastrointestinal disorders such as diarrhea, and therapy for drug dependence containing an effective amount of the peptide compounds.

### Description of the Related Art

Many peptides have been found that exhibit opiate-like activity by binding to opiate receptors. Three different types of opiate receptors have been found: delta (δ), kappa (κ) and mu (μ). The major putative function for opiates is their role in alleviating pain. Other areas where opiates are well-suited for use in treatment are conditions relating to gastrointestinal disorders, schizophrenia, obesity, blood pressure, convulsions, and seizures. Although the δ and κ receptors may also mediate analgesia, activation of μ receptors is the primary and most effective means of inducing analgesia, and is the primary mechanism by which morphine acts.

Because morphine and other compounds with clinical usefulness act primarily at the μ receptor, pharmaceutical compositions having peptides with high affinity and selectivity for this site are of considerable importance. It would be desirable to produce these peptide compositions in a simple, efficient, and economical manner.

### BRIEF SUMMARY

The subject invention provides advantageous new salts, and the free base, of mu-opiate receptor peptides. These compounds have been found to have excellent activity.

Specifically exemplified herein are acetate and trifluoroacetate salts of mu-opiate receptor peptides, and the free base of the peptides.

The peptides that can be used according to the subject invention have the general formula Tyr-X₁-X₂-X₃ wherein X₁ is Pro, D-Lys or D-Orn; X₂ is Trp, Phe or N-alkyl-Phe wherein alkyl contains 1 to about 6 carbon atoms; and X₃ is Phe, Phe-NH₂, D-Phe, D-Phe-NH₂ or p-Y-Phe wherein Y is NO₂, F, Cl or Br.

In a specific advantageous embodiment, the subject invention provides the acetate salt of a cyclic endomorphin-1 peptide (designated herein as CYT-1010).

The subject invention further provides pharmaceutical compositions comprising these advantageous peptide compounds.

The subject invention further provides therapeutic methods that utilize the salts, free base, and compositions described herein.

The subject invention further provides methods for preparing the compounds of the subject invention.

### BRIEF DESCRIPTION OF SEQUENCES

SEQ ID NO:1 is a peptide useful according to the subject invention.

SEQ ID NO:2 is a peptide useful according to the subject invention.

SEQ ID NO:3 is a peptide useful according to the subject invention.

SEQ ID NO:4 is a peptide useful according to the subject invention.

SEQ ID NO:5 is a peptide useful according to the subject invention.

SEQ ID NO:6 is a peptide useful according to the subject invention.

SEQ ID NO:7 is a peptide useful according to the subject invention.

SEQ ID NO:8 is a peptide useful according to the subject invention.

SEQ ID NO:9 is a peptide useful according to the subject invention.

SEQ ID NO:10 is a peptide useful according to the subject invention.

SEQ ID NO:11 is a peptide useful according to the subject invention.

SEQ ID NO:12 is a peptide useful according to the subject invention.

SEQ ID NOS:13-26 are additional peptides useful according to the subject invention.

### DETAILED DISCLOSURE

The subject invention provides advantageous salts of peptides, as well as the free base, that bind to the mu (morphine) opiate receptor with high affinity, selectivity and potency.

Specifically exemplified herein are acetate and trifluoroacetate (TFA) salts of mu-opiate receptor peptides, and the free base.

Advantageously, the compounds of the subject invention have excellent properties in terms of their activity.

This invention also provides pharmaceutical preparations containing an effective amount of one or more of the peptide salts and/or the free base. The subject invention further provides methods for providing analgesia, relief from gastrointestinal disorders such as diarrhea, anti-inflammatory treatments, and therapy for drug dependence wherein the methods involve administering, to a patient in need of such treatment, a composition containing an effective amount of one or more of the peptide compounds of the subject invention.

### Peptides

The peptides that can be used according to the subject invention have the general formula Tyr-X₁-X₂-X₃ wherein X₁ is Pro, D-Lys or D-Orn; X₂ is Trp, Phe or N-alkyl-Phe wherein alkyl contains 1 to about 6 carbon atoms; and X₃ is Phe, Phe-NH₂, D-Phe, D-Phe-NH₂ or p-Y-Phe wherein Y is NO₂, F, Cl or Br. Some preferred peptides of the invention are:

(SEQ ID NO: 1)
H-Tyr-Pro-Trp-Phe-NH₂
(SEQ ID NO: 2)
H-Tyr-Pro-Phe-Phe-NH₂
(SEQ ID NO: 3)
H-Tyr-Pro-Trp-Phe-OH
(SEQ ID NO: 4)
H-Tyr-Pro-Phe-Phe-OH
(SEQ ID NO: 5)
H-Tyr-Pro-Trp-D-Phe-NH₂
(SEQ ID NO: 6)
H-Tyr-Pro-Phe-D-Phe-NH₂
(SEQ ID NO: 7)
H-Tyr-Pro-Trp-pNO₂-Phe-NH₂
(SEQ ID NO: 8)
H-Tyr-Pro-Phe-pNO₂-Phe-NH₂
(SEQ ID NO: 9)
H-Tyr-Pro-N-Me-Phe-Phe-NH₂
(SEQ ID NO: 10)
H-Tyr-Pro-N-Et-Phe-Phe-NH₂
(SEQ ID NO: 11)
H-Tyr-Pro-N-Me-Phe-D-Phe-NH₂
(SEQ ID NO: 12)
H-Tyr-Pro-N-Et-Phe-D-Phe-NH₂
(SEQ ID NO: 13)
H-Tyr-c-[D-Lys-Trp-Phe]
(SEQ ID NO: 14)
H-Tyr-c-[D-Lys-Phe-Phe]
(SEQ ID NO: 15)
H-Tyr-c-[D-Orn-Trp-Phe]
(SEQ ID NO: 16)
H-Tyr-c-[D-Orn-Phe-Phe]
(SEQ ID NO: 17)
H-Tyr-c-[D-Lys-Trp-pNO₂-Phe]
(SEQ ID NO: 18)
H-Tyr-c-[D-Lys-Phe-pNO₂-Phe]
(SEQ ID NO: 19)
H-Tyr-c-[D-Orn-Trp-pNO₂-Phe]
(SEQ ID NO: 20)
H-Tyr-c-[D-Orn-Phe-pNO₂-Phe]
(SEQ ID NO: 21)
H-Tyr-c-[D-Lys-N-Me-Phe-Phe]
(SEQ ID NO: 22)
H-Tyr-c-[D-Orn-N-Me-Phe-Phe]
(SEQ ID NO: 23)
H-Tyr-c-[D-Lys-N-Et-Phe-Phe]
(SEQ ID NO: 24)
H-Tyr-c-[D-Orn-N-Et-Phe-Phe]
(SEQ ID NO: 25)
H-Tyr-c-[D-Lys-N-Me-Phe-D-Phe]
(SEQ ID NO: 26)
H-Tyr-c-[D-Lys-N-Et-Phe-D-Phe]

The last fourteen peptides listed are cyclic peptides whose linear primary amino acid sequences are given in SEQ ID NO:13 through SEQ ID NO:26. In this context, the applicants incorporate herein by reference, in its entirety, U.S. Pat. No. 6,303,578.

The peptide of SEQ ID NO:1 is highly selective and very potent for the .mu.opiate receptor, with over 4000-fold weaker binding to delta receptors and over 15,000-fold weaker binding to kappa receptors, reducing the chances of side-effects.

The peptides of this invention may be prepared by conventional solution-phase (Bodansky, M., Peptide Chemistry: A Practical Textbook, 2^{nd} Edition, Springer-Verlag, New York (1993) or solid phase (Stewart, J. M.; Young, J. D. Solid Phase Peptide Synthesis, 2^{nd} edition, Pierce Chemical Company, 1984) methods with the use of proper protecting groups and coupling agents. A suitable deprotection method may then be employed to remove specified or all of the protecting groups, including splitting off the resin if solid phase synthesis is applied.

Cyclization of the linear peptides can be performed by, for example, substitution of an appropriate diamino carboxylic acid for Pro in position 2 in the peptides through ring closure of the 2-position side chain amino and the C-terminal carboxylic functional groups. The cyclization reactions can be performed with the diphenylphosphoryl azide method (Schmidt, R., Neuhert, K., Int. J. Pept. Protein Res. 37:502-507, 1991).

Peptides synthesized with solid phase synthesis can be split off the resin with liquid hydrogen fluoride (HF) in the presence of the proper antioxidant and scavenger.

The amount of the reactants utilized in the reactions, as well as the conditions required to facilitate the reactions and encourage efficient completion may vary widely depending on variations in reaction conditions and the nature of the reactants.

The desired products may be isolated from the reaction mixture by crystallization, electrophoresis, extraction, chromatography, or other means. However, a preferred method of isolation is HPLC. All of the crude peptides can be purified with preparative HPLC, and the purity of the peptides may be checked with analytical HPLC. Purities greater than 95% of the synthesized compounds using HPLC have been obtained.

In a preferred embodiment specifically exemplified herein, the peptide is that which is shown as SEQ ID NO:13 (cyclic endomorphin-1 peptide) and has the following structure:

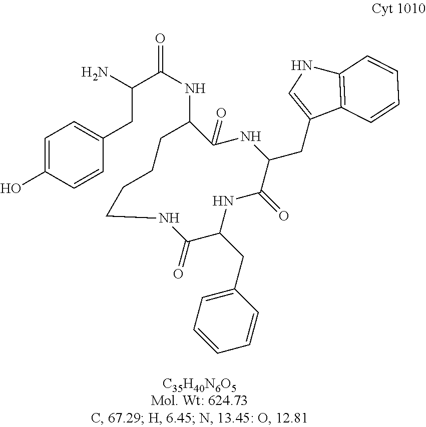 [SMILES: NC(Cc1ccc(O)cc1)C(=O)NC1CCCCNC(=O)C(Cc2ccccc2)NC(=O)C(Cc2c[nH]c3ccccc23)NC1=O]

### Pharmaceutical Compositions

The present invention also provides pharmaceutical preparations that contain a pharmaceutically effective amount of the peptide salts of this invention and a pharmaceutically acceptable carrier or adjuvant. The carrier may be an organic or inorganic carrier that is suitable for external, enteral or parenteral applications.

The peptide salts of the present invention may be compounded, for example, with the usual non-toxic, pharmaceutically acceptable carriers for tablets, pellets, capsules, liposomes, suppositories, intranasal sprays, solutions, emulsions, suspensions, aerosols, targeted chemical delivery systems (Prokai-Tatrai, K.; Prokai, L; Bodor, N., J. Med. Chem. 39:4775-4782, 1991), and any other form suitable for use. The carriers which can be used are water, glucose, lactose, gum acacia, gelatin, mannitol, starch paste, magnesium trisilicate, talc, corn starch, keratin, colloidal silica, potato starch, urea and other carriers suitable for use in manufacturing preparations, in solid, semisolid, liquid or aerosol form, and in addition auxiliary, stabilizing, thickening and coloring agents and perfumes may be used.

### Therapeutic Methods

The present invention also provides methods for providing analgesia, relief from gastrointestinal disorders such as diarrhea, and therapy for drug dependence in patients, such as mammals, including humans, which comprise administering to the patient an effective amount of the peptides, or salts thereof, of this invention. The diarrhea may be caused by a number of sources, such as infectious disease, cholera, or an effect or side-effect of various drugs or therapies, including those used for cancer therapy. For applying the peptide salts of the present invention to human, it is preferable to administer them by parenteral or enteral administration.

The peptide salts of the subject invention can also be used to provide anti-inflammatory treatments. In this context the applicants incorporate herein by reference, in its entirety, U.S. 2004/0266805.

The dosage of effective amount of the peptides varies from and also depends upon the age and condition of each individual patient to be treated. However, suitable unit dosages may be between about 0.01 to about 100 mg. For example, a unit dose may be from between about 0.2 mg to about 50 mg. Such a unit dose may be administered more than once a day, e.g. two or three times a day.

All patents, patent applications, provisional applications, and publications referred to or cited herein are incorporated by reference in their entirety to the extent they are not inconsistent with the explicit teachings of this specification.

Following is an example which illustrates aspects of the invention. This example should not be construed as limiting.

### EXAMPLE 1—ACTIVITY

The assay was the standard rat tail flick assay. Test agents were administered intravenously as suspensions in 20% PEG.

| CYT-1010 Salt form | IV Dose (mg/kg) | Activity % MPE Ave |
|---|---|---|
| Vehicle | | 1.0 |
| Acetate | 2 | 50.0 |
| | 4 | 83.3 |
| TFA | 4 | 25.4 |
| | 8 | 71.2 |
| Free Base | 2 | 77.7 |
| | 4 | 100.0 |
| HCL | 4 | 0.0 |
| Aspartate | 2 | 7.4 |
| Lactate | 2 | 1.4 |

It should be understood that the examples and embodiments described herein are for illustrative purposes only and that various modifications or changes in light thereof will be suggested to persons skilled in the art and are to be included within the spirit and purview of this application.

We claim:

1. A peptide compound wherein the compound is the acetate salt of SEQ ID NO: 13.

2. A pharmaceutical composition comprising the peptide compound of claim 1, and a pharmaceutically-acceptable carrier.

3. A method for treating a condition that is modulated by μ-opiate receptor activity, wherein said method comprises administering, to a patient in need of such treatment, the peptide compound of claim 1.

4, The method, according to claim 3, which is used to provide analgesia or to treat a condition selected from the group consisting of gastrointestinal disorders, inflammation, and drug dependence.

5. The method, according to claim 3, wherein the compound is administered enterally.

6. The method, according to claim 3, wherein the compound is administered parenterally.

7. The method, according to claim 3, wherein the composition is formulated as a solution.

8. The method, according to claim 3, comprising administering a unit dose of from 2 mg to 50 mg of the acetate salt of SEQ ID NO:13.
